(19) United States
(12) Patent Application Publication
(10) Pub. No.: US 2014/0080114 A1
(43) Pub. Date: Mar. 20, 2014

(54) METHODS, KITS AND COMPOSITIONS FOR INACTIVATION OF VIRAL AGENTS IN WHOLE BLOOD AND RED BLOOD CELL CONCENTRATES

(71) Applicant: APHIOS CORPORATION, WOBURN, MA (US)

(72) Inventors: Trevor Percival CASTOR, Arlington, MA (US); Vasudeyacharya JAYARAMA, Framingham, MA (US)

(73) Assignee: APHIOS CORPORATION, WOBURN, MA (US)

(21) Appl. No.: 14/019,671

(22) Filed: Sep. 6, 2013

Related U.S. Application Data
(60) Provisional application No. 61/697,622, filed on Sep. 6, 2012.

(51) Int. Cl. C12N 7/00

(52) U.S. Cl. CPC C12N 7/00
USPC 435/2; 435/238; 435/189; 435/283.1

(57) ABSTRACT

The present invention is directed to methods, kits and compositions for inactivating viral agents in or on articles including blood based products and feature a light sensitive compound selected from the group consisting of hypericin, pseudohypericin and hypocrellin, and a group of light producing compounds comprising luciferase, luciferin and ATP.

### RELATED APPLICATION

This application claims priority to U.S. Provisional Application No. 61/697,622, filed Sep. 6, 2012. The content of this application is hereby incorporated by reference in its entirety.

### GOVERNMENT SUPPORT

This invention was made with government support under Grant HHSN268201100034C awarded by National Institutes of Health. The government has certain rights in the invention.

### FIELD OF INVENTION

Embodiments of the present invention are directed to the preservation and safety of blood based products and red blood cell concentrates.

### BACKGROUND OF THE INVENTION

Viral infectious agents, such as, by way of example, without limitation, HIV, hepatitis B, hepatitis C, Ebola, West Nile and hantavirus, have the potential to contaminate blood supplies and blood based products. Contamination may occur in several ways; however, it is most likely to occur when blood from a blood donor infected with virus enters blood banking and blood processing systems. It is difficult to remove or inactivate viral agents potentially present in blood products without severely compromising the quality and/or the functionality of red blood cells.

There is a need to ensure blood based products, particularly, whole blood and red blood cell concentrates, are virus free.

### SUMMARY OF THE INVENTION

Embodiments of the present invention are directed to methods, kits and compositions for inactivating viral agents in or on articles including blood based products. One embodiment of the present invention, directed to a method for inactivating a viral agent, comprises the steps of placing an article potentially having a viral agent in the presence of an admixture consisting of a light sensitive compound and a group of light producing compounds. The light sensitive compound is selected from the group consisting of hypericin, pseudohypericin and hypocrellin. The group of light producing compounds is luciferase, luciferin and ATP. The light producing compounds and light sensitive compound are in a concentration for a period of time sufficient for light producing reactions to produce light sufficient to react with the light sensitive compound and the viral agent, if present, to inactivate the viral agent.

As used herein, the term “in the presence” means wetted or immersed in. By way of example, without limitation, embodiments of the present method have particular utility wherein the article is red blood cell concentrate or whole blood. The red blood cells of the whole blood or red blood cell concentrate are suspended in the admixture.

One embodiment of the present method features the light sensitive compound, hypericin, psuedohypericin and hypocrellin. These compounds are found in St. John's wort. The light sensitive compound is present in said admixture in a concentration of at least 1 micrograms per milliliter, and one embodiment features a range of between 1 micrograms per milliliter to 1000 micrograms per milliliter inclusive.

One embodiment of the present method features luciferase present in the admixture in a concentration of at least 0.10 nanoMolar, and, one embodiment features a range of 0.1 nanoMolar to 1.0 milliMolar.

One embodiment of the present method features luciferin present in the admixture in a concentration of at least 0.001 milliMolar, one embodiment features a range of 0.001 milliMolar to 10.0 milliMolar.

One embodiment of the present invention features adenosine triphosphate (referred hereinafter as “ATP”) present in said admixture in a concentration of at least 0.1 milliMolar, one embodiment features a range of 0.1 milliMolar to 1.0 Molar.

A further embodiment of the present invention features a kit for inactivating a viral agent. The term “kit” refers to a bundled group of articles and/or compositions for performing a method. For example, without limitation, one embodiment of the present invention directed to a kit comprises means for forming an admixture in an article or on an article which potentially has a viral agent and instructions. The means for forming an admixture is a light sensitive compound and a group of light producing compounds. The light sensitive compound is selected from the group consisting of hypericin, pseudohypericin and hypocrellin. The group of light producing compounds is luciferase, luciferin and ATP. The instructions are for making the admixture and designating the period of time sufficient for light producing reactions to produce light sufficient to react with the light sensitive compound and the viral agent, if present, to inactivate the viral agent.

Embodiments of the present kit have particular utility for inactivating viral agents in whole blood or red blood cell concentrate. The whole blood and red blood cell concentrate is often stored in a vessel having standard features of material composition and size. The vessel can be a sterile plastic bag normally used for storage of red blood cell concentrates. The means for forming an admixture is held in on or more vials for reconstitution in amounts to form concentrations effective for viral inactivation. The kit may further comprise one or more injection needles, spouts or such similar devices to add reconstituted admixture to vessels storing the whole blood or red blood cell concentrate.

One embodiment of the present method features the light sensitive compound, hypericin. The light sensitive compound is present in said admixture in a concentration of at least 1 microgram per milliliter, and one embodiment features a range of between 1 micrograms per milliliter to 1000 micrograms per milliliter inclusive.

One embodiment of the present method features luciferase present in the admixture in a concentration of at least 0.10 nanoMolar, and, one embodiment features a range of 0.1 nanoMolar to 1.0 milliMolar.

One embodiment of the present method features luciferin present in the admixture in a concentration of at least 0.001 milliMolar, one embodiment features a range of 0.001 milliMolar to 10.0 milliMolar.

One embodiment of the present invention features adenosine triphosphate (referred hereinafter as “ATP”) present in said admixture in a concentration of at least 0.1 milliMolar, one embodiment features a range of 0.1 milliMolar to 1.0 Molar.

A further embodiment of the present invention is directed to a composition for inactivating a viral agent. The composition comprises reconstitution means for forming an admixture in an article or on an article which potentially has a viral agent. The reconstitution means for forming an admixture consists of a solid mixture of a light sensitive compound and a group of light producing compounds. The light sensitive compound is selected from the group consisting of hypericin, pseudohypericin and hypocrellin. The group of light producing compounds is luciferase, luciferin and ATP. The reconstitution means for forming an admixture is a powder for reconstitution.

For example, without limitation, the reconstitution means for forming an admixture is a dry mixture for reconstitution of the light sensitive compound and the group of light producing compounds. The dry mixture has quantities of the light sensitive compound and the group of light producing compounds for reconstitution and application to a volume of red blood cell concentrate or whole blood.

These and other features and advantages of the present invention will be apparent to those skilled in the art upon viewing the drawing, which are briefly described in the following section, and upon reading the Detailed Description.

### BRIEF DESCRIPTION OF THE DRAWINGS

FIG. 1 depicts a kit embodying features of the present invention.

### DETAILED DESCRIPTION OF THE INVENTION

Embodiments of the present invention will now be described in detail with the understanding that the present description is considered to be the best mode now contemplated. Those skilled in the art will recognize that the best mode contemplated by the inventor may change over time and the invention is subject to modification and alteration without departing from the teaching herein. Therefore the discussion that follows should not be considered limiting.

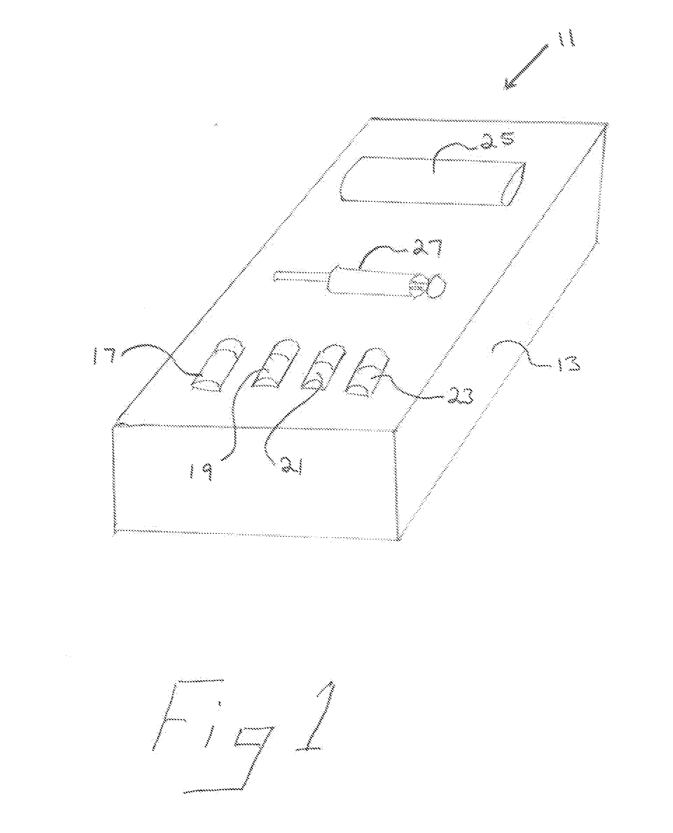

This detailed description will turn first to an embodiment of the invention directed to a kit. Turning now to FIG. 1, a kit, generally designated by the numeral 11, is depicted. Kit 11 has the following major elements for inactivating a viral agent, suitable packaging 13, a first vial 17, a second vial 19, a third vial 21, a fourth vial 23, instructions 25 and an administration tool 27. Kit 11 is a bundled group of articles and/or compositions for performing a method of viral inactivation in whole blood or red blood cell concentrate held in standard bags or bottles for collection, processing or administration.

As used herein, the term “viral inactivation” means a virion, normally capable of replication, is rendered unable to replicate. The term does not encompass filtration or separation or removal. The term encompasses performing the method where the presence or absence of virus is unknown. For example, the process is applied to whole blood or red blood cell concentrate in which the presence or absence of virus is not characterized and there is no assurance that the whole blood and red blood cell concentrate is safe for administration to an individual. Embodiments of the present invention result in a five to six log reduction in viral load. That is, the method produces a reduction in viral replication compared to control of 10,000, based on current detection limits.

Suitable packaging 13 may take several forms. The articles comprising kit 11 may be bundled by tethers, placed in bags, wrapped in flexible plastic or other wrapping material, or placed in a box or combinations of one or more known bundling methods. As depicted, suitable packaging 13 comprises a box. For purposes of clarity of the drawing, the box is not separately designated with a numeral, and this discussion will refer to suitable packaging or box interchangeably with respect to numeral 13. As depicted, box 13 holds first vial 17, second vial 19, third vial 21, fourth vial 23, instructions 25 and administration tool 27.

First vial 17, second vial 19, third vial 21, fourth vial 23 comprise means for forming an admixture in an article or on an article which potentially has a viral agent. The number of vials is arbitrary. Materials may be split into additional vials, or a vial may contain more than one composition. The first vial 17, second vial 19, third vial 21 and fourth vial 23 contain a light sensitive compound and a group of light producing compounds. For the purpose of this discussion, the first vial 17 contains a light sensitive compound. The light sensitive compound is selected from the group consisting of hypericin, pseudohypericin and hypocrellin. The Examples and this discussion will highlight hypericin. Hypericin is held in first vial 17 as a dried solid for reconstitution. First vial 17 may have opaque walls to shield the light sensitive composition from light.

The second vial 19, third vial 21 and fourth vial 23 contain light producing compounds. For example, the light producing compound luciferase is contained in second vial 19; luciferin is contained in third vial 21 and ATP is contained in fourth vial 23.

The means for forming an admixture is a light sensitive compound and a group of light producing compounds. The group of light producing compounds is luciferase, luciferin and ATP. Preferably, these light producing compounds are held as dried solids for reconstitution.

The solids for forming an admixture are held in first vial 17, second vial 19, third vial 21 and fourth vial 23 in amounts to form concentrations effective for viral inactivation in or on the article to which the admixture is applied. For example, first vial 17 holds an amount of the light sensitive compound, hypericin, to form an admixture in which the hypericin has a concentration of at least 1 micrograms per milliliter. The examples feature a range of concentrations of between about 1 micrograms per milliliter to about 1000 micrograms per milliliter inclusive.

Second vial 19 holds an amount of a compound that participates in light producing reactions, luciferase, in an amount to form concentrations, which in the presence of other light producing compounds will produce sufficient light to react the light sensitive compound with the viral agents present. The compound luciferase is in an amount to forming an admixture in which luciferase is present in the admixture in a concentration of at least 0.10 nanoMolar, and, one embodiment features a range of 0.1 nanoMolar to 1.0 milliMolar inclusive.

Third vial 21 holds an amount of a compound that participates in light producing reactions, luciferin, in an amount to form concentrations, which in the presence of other light producing compounds will produce sufficient light to react the light sensitive compound with the viral agents present. The compound luciferin is in an amount to form an admixture in which luciferin is present in the admixture in a concentration of at least 0.001 milliMolar, one embodiment features a range of 0.001 milliMolar to 10.0 milliMolar inclusive.

Fourth vial 23 holds an amount of a compound that participates in light producing reactions, ATP, in an amount to form concentrations, which in the presence of other light producing compounds will produce sufficient light to react the light sensitive compound with the viral agents present. The compound ATP is in an amount to form an admixture in which is present in the admixture in a concentration of at least 0.1 milliMolar, one embodiment features a range of 0.1 milliMolar to 1.0 Molar inclusive.

One embodiment of the present invention is directed to a solid mixture of the light sensitive compound and the group of light producing compounds which upon the addition of water are directed into the whole blood or red blood cell concentrate. The solid mixture is contained in a single vial.

The kit may further comprise one or more administration tools 27, such as an injection needle (only one is depicted) for placing water for reconstitution into the first vial 17, second vial 19, third vial 21 and/or fourth vial 23; or to place reconstituted solutions containing one or more light sensitive compounds or light producing compounds into or onto an article such as a bottle, vial or bag containing whole blood or red blood cell concentrate.

The instructions 25 describe to the user the method for making the admixture and designating the period of time sufficient for light producing reactions to produce light sufficient to react with the light sensitive compound and the viral agent, if present, to inactivate the viral agent. Other features of the instructions are to specify temperatures for performing the method, special precautions and means of disposal and storage.

Aspects of the present method are highlighted in the Examples that follow. In the Example bovine viral diarrhea virus is a model for human pathogenic viruses.

### Example 1

This example demonstrates the inactivation of bovine viral diarrhea virus (BVDV) spiked into human red blood cell concentrate (RBCC) by hypericin in the presence of chemiluminescence produced by the action of luciferase on luciferin in the presence of ATP. Ten different combinations of Dulbecco's modified eagle medium without calcium and magnesium (DMEM (—Ca++, —Mg++)), RBCC, virus, hypericin, luciferin, ATP and luciferase were prepared to all contain equivalent titers of BVDV calculated to be 6.48 log 10TCID50/mL based on the titer of the original virus stock. The concentrations of hypericin tested were 0, 40 and 200 μg/mL. Luciferase was tested at 0, 0.8 and 4.0 μM. Luciferin and ATP were present at 80 and 800 μM respectively when luciferase was present and absent in the absence of luciferase. The different combinations tested are listed in Table 1.

The components were mixed and incubated at room temperature (RT) for 2 hours in the dark followed by titration of unwashed and washed samples as previously described. Washing rows of wells with dilutions 1 and 2 was performed 3 days post-infection. Final cytopathic effects (CPEs) were read 10 days post-infection.

The various combinations tested, the titers obtained and the calculated viral reduction factors (VRFs) compared to the titer of the untreated RBC control are listed in Table 1. No clotting of RBC was observed for any of the samples during the assay. The titer of the virus stock used for spiking was 7.73 log TCID50/mL in the parallel titration. The titer of unwashed RBCC control is 6.35 logs, which is within the range of assay variation of the expected titer of 6.73 logs. However, the titer of the washed RBCC control is 3.98 logs, which is about 0.7 logs lower than expected based on the dilution factor due to washing. This is possibly due to variations from washing or due to virus inactivation at RT during the experiment.

TABLE 1
Hypericin-Luciferase Inactivation of BVDV

| | | | Luciferase | Unwashed | | Washed | | Combined |
|---|---|---|---|---|---|---|---|---|
| Sample # | Description | Hypericin | System | Titer | VRF | Titer | VRF | VRF |
| 1 | HN-LN | No | No | 6.35 | 0.00 | 3.98 | 0.00 | 2.37 |
| | (RBC | | | | | | | |
| | Control) | | | | | | | |
| 2 | HN-LL | No | Low | 6.23 | 0.12 | 3.73 | 0.25 | 2.62 |
| 3 | HN-LH | No | High | 6.48 | −0.13 | 3.10 | 0.88 | 3.25 |
| 4 | HL-LN | Low | No | 3.48 | 2.87 | <2.34 | >1.64 | >4.01 |
| 5 | HL-LL | Low | Low | 2.98 | 3.37 | <2.34 | >1.64 | >4.01 |
| 6 | HL-LH | Low | High | 3.23 | 3.12 | <2.34 | >1.64 | >4.01 |
| 7 | HH-LN | High | No | 3.10 | 3.25 | 2.48 | 1.50 | 3.87 |
| 8 | HH-LL | High | Low | 3.23 | 3.12 | 2.60 | 1.38 | 3.75 |
| 9 | HH-LH | High | High | 3.35 | 3.00 | 2.48 | 1.50 | 3.87 |
| 10 | Virus | No | No | 6.10 | NA | NA | NA | NA |
| | Control | | | | | | | |
| | (VC) | | | | | | | |

Notes:
Abbreviations:
HN, HL, HH = Hypericin-No, -Low, -High;
LN, LL, LH = Luciferase -No, -Low, -High;
NA = Not Applicable;
VRF = Virus Reduction Factor (expressed as log TCID50) = Titer of RBC control − Titer of sample;
Combined VRF = Titer of unwashed RBC control − Titer of washed sample
Titers expressed as log10TCID50/mL
Titers in yellow = <Limit of detection (LOD)
Hypericin: No = 0, low = 40 μg/mL, high = 200 μg/mL
Luciferase: No = 0, low = 0.8 μM, high = 4.0 μM
Luciferin and ATP were at 0.08 and 0.8 mM respectively for both Luciferase ‘high’ and ‘low’ but absent in ‘No’ luciferase.

There was no detectable inactivation of virus for washed samples in the absence of hypericin except for the sample treated with high dose of luciferase system where a VRF of 0.88 logs was seen. However, combined VRFs of 2.37 to 3.25 logs were seen for the three washed samples treated with no hypericin which could be explained by the reduction in titers from washing and storage at RT during the assay period. VRFs in the range of 3 logs were seen for all hypericin treated unwashed samples—both low and high doses of hypericin. The Highest VRF of 3.37 logs was seen for unwashed samples treated with low doses of both hypericin and luciferase. Low dose hypericin treatment with or without luciferase followed by washing resulted in complete elimination of the virus to undetectable levels, and appeared to be more effective than the high dose of hypericin, where residual virus was detectable in the washed samples.

These results indicate that a combination of hypericin at 40 μg/mL and luciferase at 0.8 μM was the most effective in BVDV inactivation.

### Example 2

This example demonstrates the inactivation of BVDV spiked into human RBCC by lower doses of hypericin and luciferase than those in example 1. If lower doses are effective, it would be advantageous since there will be a lower residual drug load after washing as well as lower costs per treatment.

Eleven different combinations of DMEM (—Ca++, —Mg++), RBCC, virus, hypericin, luciferin, adenosine triphosphate (ATP) and luciferase were prepared to all contain equivalent titers of BVDV calculated to be 6.48 log 10TCID50/mL (logs) based on the titer of the original virus stock. The concentrations of hypericin tested were 10, 20 and 40 μg/mL. Luciferase was tested at 0.16, 0.32 and 0.8 μM. Luciferin and ATP were present at 80 and 800 μM respectively. The different combinations tested are listed in Table 2.

The components were mixed and incubated at RT for 2 hours in the dark followed by titration of unwashed and washed samples as previously described. Washing of rows of wells with dilutions 1 and 2 was performed 2 days post-infection. Final CPE were read 10 days post-infection.

The various combinations tested, the titers obtained and the calculated VRFs compared to the titer of the untreated RBC control are listed in Table 8. There was no hemolysis or clumping for any of the samples.

TABLE 2
Hypericin-Luciferase Inactivation of BVDV

| | | | Luciferase | Unwashed | | Washed | | Combined |
|---|---|---|---|---|---|---|---|---|
| Sample # | Description | Hypericin | System | Titer | VRF | Titer | VRF | VRF |
| 1 | HL-LL | Low | Low | 5.48 | 0.50 | <2.10 | >1.63 | >3.88 |
| 2 | HL-LM | Low | Med | 4.98 | 1.00 | <2.10 | >1.63 | >3.88 |
| 3 | HL-LH | Low | High | 4.98 | 1.00 | <2.10 | >1.63 | >3.88 |
| 4 | HM-LL | Med | Low | 4.23 | 1.75 | <2.10 | >1.63 | >3.88 |
| 5 | HM-LM | Med | Med | 4.23 | 1.75 | <2.10 | >1.63 | >3.88 |
| 6 | HM-LH | Med | High | 4.10 | 1.88 | <2.10 | >1.63 | >3.88 |
| 7 | HH-LL | High | Low | 3.60 | 2.38 | <2.10 | >1.63 | >3.88 |
| 8 | HH-LM | High | Med | 3.98 | 2.00 | <2.10 | >1.63 | >3.88 |
| 9 | HH-LH | High | High | 3.48 | 2.50 | <2.10 | >1.63 | >3.88 |
| 10 | HN- | No | No | 5.98 | 0.00 | 3.73 | 0.00 | 2.25 |
| | LN(RBC | | | | | | | |
| | Control) | | | | | | | |
| 11 | Virus Stock | No | No | 7.10 | NA | ND | ND | ND |
| | (VS) | | | | | | | |

Notes:
Abbreviations:
HL, HM, HH = Hypericin-Low, -Medium, -High;
LL, LM, LH = Luciferase-Low, -Medium, -High;
NA = Not Applicable;
VRF = Virus Reduction Factor (expressed as log TCID50) = Titer of RBC control − Titer of sample;
Combined VRF = Titer of unwashed RBC control − Titer of washed sample
Titers expressed as log10TCID50/mL
Titers in yellow = <Limit of detection (LOD
Hypericin (μg/mL): low = 10, medium = 20, high = 40
Luciferase (μM): low = 0.16, medium = 0.32, high = 0.8
Luciferin and ATP were at 0.08 and 0.8 mM respectively for both Luciferase ‘high’ and ‘low’ but absent in ‘No’ luciferase.

The titer of the virus stock used for spiking was 7.10 logs in the parallel titration. The titer of unwashed RBCC control is 5.98 logs, which is within the range of assay variation of the expected titer of 6.10 logs. The titer of the washed RBCC control is 3.73 logs, which is about 0.25 logs lower than expected based on the dilution factor due to washing. This is within the range of assay variation but also could be possibly due to variations from washing or due to virus inactivation at RT during the experiment.

There was complete inactivation/elimination of virus for washed samples for all the levels of treatment with a VRF of >1.63 logs. The combined VRFs for these samples were >3.88 logs. The VRFs for unwashed samples showed a dose response to the levels of hypericin ranging from 0.5 to 1 logs for low, 1.75 to 1.88 logs for medium and 2.0 to 2.50 logs for high. For each level of hypericin there also appeared to be a dose dependency for the levels of luciferase, although the differences were small enough that they could be due to assay variation. The highest level of inactivation was seen with a combination of hypericin at 40 μg/mL and luciferase at 0.8 μM.

### Example 3

This example demonstrates the inactivation of BVDV spiked into human RBCC by hypericin in doses ranging from 40 to 120 μg/mL in the presence of different doses of luciferase. This dose range had not been tested in the previous experiments. Ten different combinations of DMEM (—Ca++, —Mg++), RBCC, virus, hypericin, luciferin, adenosine triphosphate (ATP) and luciferase were prepared to all contain equivalent titers of BVDV calculated to be 6.48 log 10TCID50/mL (logs) based on the titer of the original virus stock. The concentrations of hypericin tested were 40, 80 and 120 μg/mL. Luciferase was tested at 0.16, 0.32 and 0.8 μM. Luciferin and ATP were present at 80 and 800 μM respectively. The different combinations tested are listed in Table 3.

TABLE 3
Hypericin-Luciferase Inactivation of BVDV

| | | | Luciferase | Unwashed | | Washed | | Combined |
|---|---|---|---|---|---|---|---|---|
| Sample # | Description | Hypericin | System | Titer | VRF | Titer | VRF | VRF |
| 1 | HL-LL | Low | Low | 3.85 | 2.00 | <2.34 | >1.14 | >3.51 |
| 2 | HL-LM | Low | Med | 3.35 | 2.50 | <2.34 | >1.14 | >3.51 |
| 3 | HL-LH | Low | High | 3.35 | 2.50 | <2.34 | >1.14 | >3.51 |
| 4 | HM-LL | Med | Low | 2.98 | 2.88 | 3.10 | 0.38 | 2.75 |
| 5 | HM-LM | Med | Med | 3.10 | 2.75 | 3.10 | 0.38 | 2.75 |
| 6 | HM-LH | Med | High | 2.98 | 2.88 | 3.10 | 0.38 | 2.75 |
| 7 | HH-LL | High | Low | 3.23 | 2.63 | 3.10 | 0.38 | 2.75 |
| 8 | HH-LM | High | Med | 3.23 | 2.63 | 3.10 | 0.38 | 2.75 |
| 9 | HH-LH | High | High | 2.48 | 3.38 | 3.10 | 0.38 | 2.75 |
| 10 | HN- | No | No | 5.85 | 0.00 | 3.48 | 0.00 | 2.38 |
| | LN(RBC | | | | | | | |
| | Control) | | | | | | | |
| 11-L | Virus | No | No | 5.85 | NA | ND | ND | ND |
| | Control | | | | | | | |
| 11-R | Virus Stock | No | No | 6.98 | NA | ND | ND | ND |

Notes:
Abbreviations:
HL, HM, HH = Hypericin-Low, -Med, -High;
LL, LM, LH = Luciferase-Low, Med, -High;
NA = Not Applicable;
VRF = Virus Reduction Factor (expressed as log TCID50) = Titer of RBC control − Titer of sample;
Combined VRF = Titer of unwashed RBC control − Titer of washed sample
Titers expressed as log10TCID50/mL
Titers in yellow = <Limit of detection (LOD)
Hypericin doses (μg/mL)-Low = 40, Med = 80, High = 120
Luciferase (μM)-Low = 0.16, Med = 0.32, High = 0.8
Luciferin and ATP were at 0.08 and 0.8 mM respectively for both Luciferase ‘high’ and ‘low’ but absent in ‘No’ luciferase.

The compounds were mixed and incubated at RT for 2 hours in the dark followed by titration of unwashed and washed samples as previously described. Washing rows of wells with dilutions 1 and 2 was performed 2 days post-infection. Final CPEs were read 10 days post-infection.

The various combinations tested, the titers obtained and the calculated VRFs compared to the titer of the untreated RBC control are listed in Table 9. There was no hemolysis or clumping for any of the samples at the end of the treatment period.

The titer of the virus stock used for spiking was 6.98 logs in the parallel titration. The titer of unwashed RBCC control is 5.85 logs, which is within the range of assay variation of the expected titer of 5.98 logs. The titer of the washed RBCC control is 3.48 logs, which is about 0.37 logs lower than expected based on the dilution factor due to washing. This is within the range of assay variation.

There was complete inactivation/elimination of virus for washed samples for all the levels of luciferase at a hypericin dose of 40 μg/mL with a VRF of >1.14 logs. The combined VRFs for these samples were >3.51 logs. However, complete inactivation/elimination was not seen for the washed samples at higher doses of hypericin where true residuals were observed.

These results indicate that of the doses tested, hypericin at 40 μg/mL is the most effective for all three doses of luciferase tested. Higher VRF was seen for hypericin-luciferase doses at the high level for each but the washed RBCs showed a true residual in this case. In summary, the above results indicate that the optimal dose of hypericin is 40 μg/mL with different doses of luciferase for BVDV.

Thus, embodiments of the present invention have been described in detail with the understanding that the present description is directed to the best mode presently contemplated. However, the present invention, as described, is capable of modification and alteration and the description should not be limiting but should encompass the subject matter of the claims that follow and their equivalents.

1. A method for inactivating a viral agent comprising the steps of placing an article potentially having a viral agent in the presence of an admixture consisting of a light sensitive compound and a group of light producing compounds, wherein said light sensitive compound is selected from the group consisting of hypericin, pseudohypericin and hypocrellin, said group of light producing compounds is luciferase, luciferin and ATP, said light producing compounds and light sensitive compound in a concentration for a period of time sufficient for light producing reactions to produce light sufficient to react with said light sensitive compound and said viral agent to inactivate said viral agent.

2. The method of claim 1 wherein said article is red blood cell concentrate.

3. The method of claim 1 wherein said article is whole blood.

4. The method of claim 1 wherein said light sensitive compound is hypericin.

5. The method of claim 1 wherein said light sensitive compound is present in said admixture in a concentration of at least 1 micrograms per milliliter.

6. The method of claim 5 wherein said light sensitive compound is present in a range of between 1 micrograms per milliliter to 1000 micrograms per milliliter inclusive.

7. The method of claim 1 wherein luciferase is present in said admixture in a concentration of at least 0.10 nanoMolar.

8. The method of claim 7 wherein said luciferase is present in a range of 0.1 nanoMolar to 1.0 milliMolar.

9. The method of claim 1 wherein said luciferin is present in said admixture in a concentration of at least 0.001 milliMolar.

10. The method of claim 9 wherein said luciferin is present in said admixture in a concentration range of 0.001 milliMolar to 10.0 milliMolar.

11. The method of claim 1 wherein said ATP is present in said admixture in a concentration of at least 0.1 milliMolar.

12. The method of claim 9 wherein said ATP is present in said admixture in a concentration range of 0.1 milliMolar to 1.0 Molar.

13. A kit for inactivating a viral agent comprising:
means for forming an admixture in an article or on an article which potentially has viral agent/s and instructions, said means for forming an admixture consisting of a light sensitive compound and a group of light producing compounds, wherein said light sensitive compound is selected from the group consisting of hypericin, pseudohypericin and hypocrellin, said group of light producing compounds is luciferase, luciferin and ATP wherein said instructions for making said admixture and designating the period of time sufficient for light producing reactions to produce light sufficient to react with light sensitive compound and said viral agent to inactivate said viral agent.

14. The kit of claim 13 wherein said article is whole blood or red blood cell concentrate held in a vessel.

15. The kit of claim 13 wherein said light sensitive compound is hypericin.

16. The kit of claim 13 wherein said means for forming an admixture is held in on or more vials for reconstitution.

17. The kit of claim 16 further comprising one or more injection needles for adding said light sensitive compound, luciferase, luciferin and ATP to said article.

18. The kit of claim 13 wherein said light sensitive compound is present in said admixture in a concentration of at least 1 micrograms per milliliter.

19. The kit of claim 18 wherein said light sensitive compound is present in a range of between 1 micrograms per milliliter to 1000 micrograms per milliliter inclusive.

20. The kit of claim 13 wherein luciferase is present in said admixture in a concentration of at least 0.10 nanoMolar.

21. The kit of claim 13 wherein said luciferase is present in a range of 0.1 nanoMolar to 1.0 milliMolar.

22. The kit of claim 13 wherein said luciferin is present in said admixture in a concentration of at least 0.001 milliMolar.

23. The method of claim 9 wherein said luciferin is present in said admixture in a concentration range of 0.001 milliMolar to 10.0 milliMolar.

24. The kit of claim 13 wherein said ATP is present in said admixture in a concentration of at least 0.1 milliMolar.

25. The method of claim 9 wherein said ATP is present in said admixture in a concentration range of 0.1 milliMolar to 1.0 Molar.

26. A composition for inactivating a viral agent comprising:
means for forming an admixture in an article or on an article which potentially has a viral agent, said means for forming an admixture consisting of a light sensitive compound and a group of light producing compounds, wherein said light sensitive compound is selected from the group consisting of hypericin, pseudohypericin and hypocrellin, said group of light producing compounds is luciferase, luciferin and ATP wherein said means for forming an admixture is a powder for reconstitution.

27. The composition of claim 26 wherein said means for forming an admixture is a dry mixture for reconstitution of said light sensitive compound and said group of light producing compounds.

28. The composition of claim 27 wherein said dry mixture has quantities of said light sensitive compound and said group of light producing compounds for reconstitution and application to a volume of red blood cell concentrate or whole blood.
